(19) United States
(12) Patent Application Publication
(10) Pub. No.: US 2018/0168213 A1
(43) Pub. Date: Jun. 21, 2018

(54) Growing Anti-Pathogen Microbes Found In Raw Seeds To Sufficient Numbers Inside An Organic Mixture Or Culture Medium So That The Entire Mixture Could Then Be Used As A Type Of All-Natural Infectious Diseases Immunotherapy

(71) Applicant: Larry Lumpkin, JR., Grand Terrace, CA (US)

(72) Inventor: Larry Lumpkin, JR., Grand Terrace, CA (US)

(73) Assignee: Larry (NMI) Lumpkin, JR., Grand Terrace, CA (US)

(21) Appl. No.: 15/387,165

(22) Filed: Feb. 22, 2017

(51) Int. Cl. A23L 33/135; A23L 33/105; A61K 35/74; A61K 45/06

(52) U.S. Cl. CPC A23L 33/135; A61K 45/06; A61K 35/74; A23L 33/105

(57) ABSTRACT

A product and method that uses the ideal raw, plant-based, nutrient-rich, organic mixture to act as the perfect culture medium which allows and encourages recently discovered, continuously evolving, and extremely small but very powerful anti-pathogen microbes found in raw seeds to rapidly grow and multiple and to sufficient numbers so that these microbes and the plant-based mixture that comprises them can then be used together as an effective type of all-natural Infectious Diseases Immunotherapy.

### CROSS REFERENCE TO RELATED APPLICATION

This application is a continuation of provisional application No. 60/386,040, filed Nov. 17, 2015 which is incorporated by reference herein in its entirety for all purposes.

### STATEMENT REGARDING FEDERALLY SPONSORED RESEARCH OR DEVELOPMENT

“Not Applicable”

### REFERENCE TO A SEQUENCE LISTING, A TABLE, OR A COMPUTER PROGRAM, LISTING COMPACT DISC APPENDIX

“Not Applicable”

### FIELD OF THE INVENTION

This application relates to growing powerful anti-pathogen microbes found in raw seeds to sufficient numbers inside the ideal plant-based organic mixture so that the entire mixture can then be used as a type of all-natural Infectious Diseases Immunotherapy.

### BACKGROUND INFORMATION

Each year, billions of dollars are spent on new and existing drugs, vaccines and other treatments and methods to help suppress, contain, control and/or neutralize many types of existing, emerging and reemerging infectious diseases. But when the vaccines, drugs and other medical procedures aren't working or don't exist, the only thing we have left to rely on is a strong, healthy, smart, sharp immune system. And no matter how bad an outbreak, epidemic or pandemic gets, there always a larger percentage of the population that can completely resist or quickly dispense of these infections without any or very little medical treatment, and that's because these people often have immune systems which are strong, healthy and smart enough to promptly take care of these infections.

Immunotherapies are quickly becoming the newest way to fight many types of cancers, but these therapies have been erratic in their results and are extremely complicated and expensive to perform on patients. The applicant believes he has also developed a new type of immunotherapy but to help fight infectious diseases, and this therapy had an extremely high success rate during a three phase clinical trial and is quite easy and inexpensive to make and use on patients.

Now, the planet earth has a marvelous and very complex ecosystem, and the five most important elements of our ecosystem are probably the sun, our atmosphere, water, soil and seeds. Our ecosystem also contains plenty of continuously evolving pathogens whose primary function is to help keep everything in check. But even with its billions or trillions of pathogens, the earth's ecosystem was not designed to destroy itself. This of course means seeds, which directly or indirectly are the most basic source of nutrients for every single living organism in our ecosystem, are programmed to be and are being thoroughly protected with continuously evolving innate and active defenses which help them quickly adjust to and/or stay several steps ahead of most if not all of the evolving natural or man-made pathogens which might threaten their existence.

One of the evolving active defenses the applicant believes seeds use are never discovered before, extremely small (possibly smaller than viruses) but very powerful anti-pathogen microbes which, under ideal conditions, can help seeds survive for hundreds of years before being planted, and then, once planted, help thoroughly protect the small embryo inside the seeds from the many different natural and/or man-made pathogens they often get exposed to during the delicate germination process.

The applicant believes the main problem or drawback with the anti-pathogen microbes inside raw seeds is not only are they too small to currently be detected, but they are also insufficient in numbers to be beneficial to the human body and its immune system in any way.

So, where's the scientific evidence that supports the applicant's claim that these powerful anti-pathogen microbes inside raw seeds actually exist, and, if so, that they can be used to help the cells of the human immune system do a much better job of fighting infectious diseases? Well, please remember that the virus was discovered or theorized in or about 1898 but wasn't actually seen until 1931 upon the invention of the electron microscope, and it is widely accepted that scientists have only discovered and cataloged about 15% of the vast number of microorganisms in our ecosystem.

For this nutritional supplement to function effectively as an all-natural Infection Diseases Immunotherapy that's cheap and easy to make with minimal side-effects, it has to be made with safe to consume raw seeds and the ideal and safe to combine combinations of raw, nutrient-rich, plant-based ingredients.

The nutritional supplement is designed to provide the ideal plant-based nutrient-rich environment that allows and encourages the anti-pathogen microbes inside one or a blend of safe to consume raw seeds added to the mixture to rapidly grow and multiply and to sufficient numbers to be beneficial to the cells of the human immune system when these cells are fighting many types of infectious diseases.

The nutritional supplement is designed to help promptly but gently get rid of the too many new and old foreign invaders, toxins and pockets of inflammation inside the body that often take too much time and energy away from the cells of the immune system which could be used to help fight infectious diseases.

The nutritional supplement is designed to help quickly and naturally treat the general signs and symptoms of many infectious diseases such as nausea, vomiting, fever, diarrhea, fatigue, muscle and body aches and pain, congestion and coughing.

The nutritional supplement is designed to help quick supply the gut's micro-flora with vital micro-nutrients which are often just as important and effective as probiotics in helping to balance the intestinal bacterial flora and keeping the digestive tract at a pH level that makes it almost impossible for bacteria to take root and grow.

The nutritional supplement is designed to reduce, as much and as fast as possible, the SIBO or small intestinal bacterial overgrowth by helping to greatly increase the amount of stomach acid going into the small intestines.

The nutritional supplement is designed to quickly supply the cells of the immune system, 80% of which are located in the gut, with a thick, rich but safe mass of vital nutrients needed by these cells to function at optimum levels.

The nutritional supplement is designed to use anti-pathogen microbes found in raw seeds to help get rid of, as much and as fast as possible, any contaminates and/or viral infections from around and/or inside human immune cells which may be hindering, weakening and/or suppressing their performance, destroying them and/or causing some type of autoimmune disorder.

The nutritional supplement is designed to use raw seed-based anti-pathogen microbes to help assist the cells of the immune system in doing a much more efficient, faster and thorough job of completely clearing away many types of infectious diseases from the human body, and these microbes may also be effective in going after those infections that like to hide or linger in parts of the body that aren't entirely under the control of the immune system or infections that are able to go dormant inside cells so that the cells of the immune system can't detect or find them.

### DETAILED DESCRIPTION OF THE INVENTION

The present invention provides a method for growing anti-pathogen microbes found in safe to consume raw seeds to sufficient numbers inside the ideal plant-based organic mixture so that the entire mixture could then be used to help the cells of the human immune system do a much more effective, faster and/or thorough job of suppressing, containing, neutralizing and/or completely clearing from the body many types of infectious diseases, and usually before any serious complications can occur. To be considered an effective type of Infectious Diseases Immunotherapy, the organic mixture is designed to provide the perfect nutrient rich environment to allow and encourage these seed-based anti-pathogen microbes to rapidly grow and multiply, to remove as many foreign invaders and pockets of inflammation as possible from the body, to balance the gut's intestinal bacterial flora, to get rid of as much and as fast as possible any small intestinal bacteria overgrowth, to quickly supply the cells of the human immune system with the needed vital nutrients, to help clear the cells of the immune system from any contaminants or viral infections from around or inside these cells and to help assist the cells of the human immune system in doing a much more effective, faster and thorough job of doing what these cells are suppose to do.

The nutritional supplement or organic mixture is designed to allow and encourage raw seeds' anti-pathogen microbes to rapidly grow and multiply by supplying these microbes with an abundant but safe amount of the nutrients or stored foods they survive on inside the seed. Inside seeds, food is stored in parts of the seed known as the endosperm, cotyledons or perisperm, and this stored food consists of carbohydrates, fats and proteins. For every 64 ounces of this organic mixture, at least two-thirds cup of a single or a combination of safe to consume raw seeds plus one or more seeds from an avocado will be blended into the mixture to help provide the ideal nutrients for these microbes to rapidly grow and multiply with other carbohydrates, fats, proteins and other vital nutrients coming from other possible ingredients such as a plant-based protein powder, a safe combination of raw organic fruits and vegetables from all five color groups, and granular kelp and other spices. All the ingredients of the mixture are blended together using a liquid base of either water or ice, coconut water, a no-sugar-added fruit juice or some other plant-based liquid and a powerful commercial blender with an at least 3 horsepower motor in order to achieve the desired consistency to help these microbes grow. The final blended mixture is poured into a 64 oz bottle, sealed and stored in a refrigerator for 6 to 12 hours before usage. The time of storage and the space of the 64 oz sealed container somehow convinces these microbes they now have one big giant seed to protect, so microbes promptly start using the abundant but safe amount of added vital nutrients to rapidly grow and multiply.

The nutritional supplement or organic mixture is designed to promptly but gently remove as many foreign invaders and pockets of inflammation as possible from the body. These foreign invaders mostly come from the foods and legal and illegal drugs consumed and often cause a lot of inflammation and take too much time and energy away from the cells of the immune system which could be used to help fight infectious diseases. To accomplish this goal, the organic mixture is loaded with cleansing fibers and nutrients which come from the ideal and safe combination of raw, organic fruits and vegetable and spices such as cinnamon, cardamom, granular kelp and others, and the organic mixture must be the only food consumed during the first 24 hours of usage or treatment, if at all possible, along with a little water while following recommended dosages.

The nutritional supplement or organic mixture is designed to help balance the gut's intestinal bacterial flora. One of the ways infectious diseases often use to establish themselves in the human body is to quickly deprive the cells of the immune system of the vital nutrients they need to function at optimum levels. These infections do this by causing lots of nausea, vomiting, diarrhea, loss of appetite and by throwing the body's bacteria flora out of balance, causing an immediate buildup of bad bacteria in the small intestines which in turn block or limit vital nutrients from being absorbed by the cells of the immune system and the entire body. This is why these infections sometimes cause so much weakness, malaise, body aches and fatigue. The organic mixture will use the ideal mixture of fresh, raw, organic fruits and vegetables, seeds and spices to quickly provide the gut with a safe but abundant amount of minerals such as manganese, phosphorus, magnesium, copper and zinc which have proven to work just as well and as fast as probiotics in helping to balance the gut's bacterial flora. A balanced bacterial flora has the ability to prevent pathogenic organisms from seeping out of the GI tract and into the bloodstream, possibly causing some serious complications.

The nutritional supplement is a thick rich organic mixture that's blended using a powerful commercial blender to a consistency very similar to that of digested food in the stomach. This of course means the mixture will spend a limited amount of time in the stomach, usually between 1 to 2 hours, and during this time it will use ingredients such as whole lemon juice (juice from juicing whole lemons), pineapples and spices to to help increase the production of stomach acid without causing indigestion or acid reflux, and this additional stomach acid will be passed into the small intestines along with the digested mixture. A consistent large amount of stomach acid in the small intestines has proven to work just as well and as fast as probiotics and even antibiotics in helping to reduce the amount of bad bacteria overgrowth in the small intestines, thus allowing the cells of the immune system to receive more of the vital nutrients needed to effectively fight infectious diseases and the rest of the cells in the body to receive the vital nutrients needed to help fight infection symptoms such as fever, fatigue, body aches, malaise and loss of appetite.

The nutritional supplement or organic mixture is designed to quickly supply the cells of the immune system with a massive but safe amount of the vital raw nutrients these cells need to function at optimum levels while, at the same time, using the anti-pathogen microbes inside the mixture to help clear away contaminates and viral infections from around and/or inside immune cells and then help these immune cells do a much more effective, faster and thorough job of completely clearing the body of many types of infectious diseases. With the bad bacteria in the small intestines out of the way, as much as possible, the cells of the immune system can now readily absorb vital nutrients such as vitamins A, B, C and E and the mineral zinc from the ideal and safe to consume combination of fresh, raw, organic fruits, vegetables, raw seeds and spices in the organic mixture. At the same time, the cells of the immune system will also be absorbing the anti-pathogen microbes inside the mixture, and these microbes will quickly get to work helping to clear away, if at all possible, any contaminants and/or viral infections from around and/or inside these immune cells which may be hindering, weakening and/or suppressing the performance of these immune cells, destroying them and/or causing some type of autoimmune disorder. The anti-pathogen microbes will then help the newly energized and now hopefully healthy immune cells locate and/or identify infections in the body where ever they might be so that these infections can be, if at all possible, completely cleared from the body in a timely manner, usually within 2 to 5 days, and before any serious complications can occur and with minimal side effects from consuming the organic mixture.

The nutritional supplement or organic mixture qualifies as an all-natural type of Infectious Diseases Immunotherapy because it has been specifically designed and proven to help the cells of the human immune system do a much more effective, efficient, faster and thorough job of suppressing, containing, neutralizing and/or completely clearing from the body many types of infectious diseases by using carbohydrates, fats and proteins from foods such as raw seeds, granular kelp, raw organic fruits and vegetables and various spices to help allow and encourage the extremely small and undetectable anti-pathogen microbes inside safe to consume raw seeds to rapidly grow and multiply and to sufficient numbers so that these microbes can be used to help clear the cells of the immune system from any contaminants and/or viral infections which may be adversely affecting these cells' performance, by using cleansing fibers and nutrients from such ingredients as leafy greens, other raw organic fruits and vegetables and spices such as cinnamon, cardamom and others to help clear the system within 10 to 12 hours and as much as possible of any foreign invaders and pockets of inflammation which may be taking too much time and energy away from the immune cells, by using abundant but safe amounts of minerals such as manganese, phosphorus, magnesium, copper and zinc from raw seeds, various spices and the ideal but safe combination or organic fruits and vegetables to help balance the gut's bacterial flora as fast as possible and thereby help prevent infections from seeping into the bloodstream, by using such ingredients and fresh whole lemon, pineapples, spices and others to help produce and then send a constant large amount of stomach acid into the small intestines to help control small intestinal bacteria overgrowth and thereby help stop this overgrowth from blocking or limiting vital nutrients from reaching the immune cells and by quickly supplying the cells of the immune system with a thick, rich, massive but safe amount of vital nutrients such as vitamins A, B, C and E and the mineral zinc from such fresh raw ingredients as seeds, organic fruits and vegetables so that the immune cells can function at optimum levels.

The nutritional supplement or organic mixture works best when raw, organic fruits and/or vegetables from the six different color groups (green, white, red, orange, yellow and blue/purple) are included in the mixture because each color contains different vital nutrients. While only one type of seed can be used in the mixture along with one or more avocado seeds, it's best to use a blend of safe to consume and compatible raw seeds because each seed contains varying amounts of vital nutrients needed to make the organic mixture perform as claimed. Various compatible spices should also be used because they too contain varying amounts of needed vital nutrients.

The nutritional supplement or organic mixture can be made from varying different combinations of fruits, vegetable, seeds and spices, but these combinations must be ideal and compatible and safe to consume or the mixture can cause some serious side-effects such as indigestion and acid reflux, excessive bloating, cramping and gas, diarrhea and/or constipation and even organ damage or failure. All the ingredients that are chosen to be used should be blended together using a powerful commercial blender so that the mixture can reach the desired consistency to perform as claimed. The finished blended product should be poured into a 64 oz bottle, sealed and allowed to sit in a refrigerator for 6 to 24 hours so that the anti-pathogen microbes inside the seeds can rapidly grow and multiply to the sufficient numbers needed for the mixture to be effective. The overall mixture works best when it is made fresh when ordered, no unnatural chemicals, additives or preservatives (whole lemon juice is the only preservative) are added, the recommended dosages are taken, the entire contents is consumed within 10 days after blending and the user limits or completely avoids the consumption of caffeine, artificial sweeteners, dairy, alcohol and products containing white or wheat flour during the entire treatment period of 2 to 5 days.

The nutritional supplement or organic mixture proved its effectiveness as an all-natural type of Infectious Diseases Immunotherapy during a three-phase clinical trial that included over three thousand hospitalized patients. The organic mixture was originally created to only be a just as good or more effective and safer to use alternative to the most popular prescribed and over-the-counter cold and influenza antiviral medications, which it proved to be during the clinical trial. In fact, during this clinical trial, doctors were so impressed with how well the organic mixture performed against various types of cold and influenza viruses and some of their complications such as influenza associated pneumonia, bronchitis and sinusitis that the doctors decided to test the organic mixture on patients suffering from other infectious diseases such as measles, mumps, rota-virus, strep throat, gastroenteritis, various sexually transmitted diseases, the Zika virus and other diseases with varying but mostly satisfactory results. The organic mixture also helped enhance the performance of some antiviral and antibiotic drugs. Many elderly patients given the organic mixture during the clinical trial reported a marked increase in their energy and vitality levels, a marked reduction in chronic pain and gastrointestinal issues and more mobility and less swelling and pain in arthritic joints. The only documented negative side-effects of the organic mixture were a slight to moderate increase in lower intestinal gas and a softer (not diarrhea) more frequent stool.

Remember, the best way to fight and defeat any type of infectious disease is with a strong, healthy, energized and smart immune system, and the organic mixture described in this application is designed and has proven to help cleanse, nourish, strengthen, energize and assist the cells of the human immune system in doing a much more effective, efficient, faster and thorough job of doing what they are suppose to do when fighting infectious diseases.

### DRAWINGS

“Not Applicable”

### SEQUENCE LISTING

“Not Applicable”

### OATH OR DECLARATION

1. A nutritional supplement product designed to be an all-natural Infectious Diseases Immunotherpy comprising: the ideal, raw, plant-based, nutrient-rich, organic mixture that acts as the perfect culture medium which allows and encourages powerful anti-pathogen microbes recently discovered by applicant in raw seeds to rapidly grow and multiply and to sufficient numbers, usually within 6 to 24 hours when the final mixture is stored in a refrigerator, so that these microbes and the raw, plant-based mixture that comprises them can then both be useful to the cells of the human immune system when these cells are fighting many types of infectious diseases.

2. A nutritional supplement product designed to be an all-natural Infectious Diseases Immunotherapy according to claim 1, wherein the nutritional supplement helps to promptly but gently flush or clear away, as much and as fast as possible, the too many foreign invaders and pockets of inflammation in the human body system, usually from consumed food products and/or legal and/or illegal drugs, which often preoccupy too much time and energy away from the cells of the human immune system which could be used to help fight infectious diseases.

3. A nutritional supplement product designed to be an all-natural Infectious Diseases Immunotherapy according to claim 1, wherein the nutritional supplement helps quickly balance the human body's intestinal bacteria flora, while at the same time helping to clear away, as much and as fast as possible, massive amounts of bad bacteria inside the small intestines which often block or limit the absorption of vital nutrients needed by the cells of the human immune system to operate at optimum levels.

4. A nutritional supplement product designed to be an all-natural Infectious Diseases Immunotherapy according to claim 3, wherein once the nutritional supplement has cleared away, as much as possible, the bad bacteria from the small intestines, the supplement then quickly supplies the cells of the immune system and of the entire body with a thick, rich, mass of raw, plant-based, energizing nutrients, while at the same time the anti-pathogen microbes inside the mixture go to work helping to clear away, as much and as fast as possible, any contaminates and/or viral infections from around and/or inside the cells of the immune system which may be hindering, weakening or suppressing these cells' performance, destroying them and/or causing some types of autoimmune disorders. These seed based anti-pathogen microbes will also help assist the cells of the human immune system in clearly away many types of viral and bacterial infections completely from the body, especially those infections that like to linger and/or hide, and usually before any serious complications can occur.

5. A nutritional supplement product designed to be an all-natural Infectious Disease Immunotherapy according to claim 1, wherein the nutritional supplement's effectiveness in helping the human immune system do a much more effective, faster and thorough job of containing, suppressing, neutralizing and/or completely clearing from the body usually within 2 to 5 days a number of infectious diseases and primarily influenza and cold viruses and their complications, was demonstrated and proven during a three phase clinical trial which had over 3,000 hospitalized patients taking a recommended dosage of the nutritional supplement tor a period of two to five days with a 75 to 100 percent success rate, depending on the infectious disease, and with the only recorded negative side-effects being a slight to moderate increase in lower intestinal gas and a softer (not diarrhea) and more frequent stool.
